(19) United States
(12) Patent Application Publication
(10) Pub. No.: US 2006/0260188 A1
(43) Pub. Date: Nov. 23, 2006

(54) Vegetation block and outer layer body for the same

(75) Inventor: Higashi Ko, Iwate (JP)

Correspondence Address: KANESAKA BERNER AND PARTNERS LLP, SUITE 300, 1700 DIAGONAL RD, ALEXANDRIA, VA, 22314-2848, US

(73) Assignee: SEINAN INDUSTRY CORPORATION, Iwate (JP)

(21) Appl. No.: 10/551,390

(22) PCT Filed: Jun. 15, 2004
(86) PCT No.: PCT/JP04/08675

§ 371 Date: Sep. 29, 2005

(51) Int. Cl. A01H 3/00

(52) U.S. Cl. 47/58.1 R

(57) ABSTRACT

A vegetation block comprises a block main body 10 made of a concrete, and an outer layer body 20 constituted by forming, into a mat shape, a fiber aggregate F which is located on an outer surface of the block main body 10 during the forming of the block main body 10 and which enables growth of a plant. The outer layer body 20 is formed by inserting many needles into the stacked fiber F from the upper side thereof and then pressing the needles, and many longitudinal fibers are allowed-to be present in the fiber aggregate F of the outer layer body 20 to facilitate entry of the fibers into the concrete. Moreover, aggregates of uniform grain. sizes are used as aggregates of the concrete, and a porosity of the concrete is set to be 10 to 25% so that the fibers of the outer layer body 20 may easily enter into the concrete. In consequence, the outer layer body 20 is surely bonded to the concrete so as to prevent the outer layer body 20 from easily peeling off, and the rooting of the grown plant in the fiber aggregate surely proceeds.

### TECHNICAL FIELD

The present invention relates to a vegetation block which is installed on a slope along a road, a bank protection or the like and which enables growth of a plant, and an outer layer body for the vegetation block.

### BACKGROUND ART

Conventionally, as a vegetation block of this type, for example, there has been known a vegetation block previously proposed by the applicant of the present invention and others and described in Japanese Patent Application Laid-Open No. 2002-220836.

As shown in FIG. 11, a vegetation block Ba is configured by comprising a block main body 1 formed out of concrete and having a bottom surface 2 installed in an installation surface, an outer layer body 4 constituted by forming, into a sheet shape, a fiber aggregate F which is located on an outer surface 3 excluding the bottom surface 2 of the block main body 1 and which enables growth of a plant, and a frame body 5 of, e.g., a lattice shape for reinforcement which is disposed on an inner surface of the outer layer body 4. Reference numeral 6 denotes connecting metal fittings which outwardly project from the block main body 1 and which are connectable to the adjacent block main body 1.

In the case of manufacturing the vegetation block Ba, the outer layer body 4 and the frame body 5 are first formed into shapes similar to a shape of the other surface 3 of the block main body 1, and the outer layer body 4 is bonded to the frame body 5. Then, the outer layer body 4 is attached together with the frame body 5 to a die surface of a forming die (not shown) of the block main body 1, and concrete is subsequently poured into the forming die and then formed.

Meanwhile, in the conventional vegetation block Ba, the outer layer body 4 is attached to the block main body 1 through the lattice-shaped frame body 5. However, the frame body 5 and fibers of the fiber aggregate F are not always well intertwined with and bonded to the block main body 1, and hence there is a problem that the fiber aggregate F is easily peeled off. Furthermore, even if it is intended that the fiber aggregate F of the outer layer body 4 is directly bonded to the block main body 1 without disposing the frame body 5, the fibers of the fiber aggregate F are not necessarily well bonded to the block main body 1, and also in this case, the fiber aggregate F may easily be peeled off. In consequence, even when the plant grows in the fiber aggregate F, the rooting of the plant may be insufficient. In the case that the vegetation block is used on a bank protection of a river, the fiber aggregate is easily run off by increased water of the river or a rainstorm, which may make it impossible to secure a satisfactory growth environment of the plant.

The present invention has been developed in view of such problems, and an object of the present invention is to provide a vegetation block which can secure a satisfactory growth environment of a plant by bonding a fiber aggregate to a block main body so as to prevent the fiber aggregate from peeing off and by assuring the rooting of the grown plant in the fiber aggregate. Another object of the present invention is to provide an outer layer body for the vegetation block.

### DISCLOSURE OF THE INVENTION

To solve the problems, a vegetation block of the present invention comprises a block main body formed out of concrete, and an outer layer body constituted by forming, into a mat shape, a fiber aggregate which is located on an outer surface of the block main body during the forming of the block main body and which enables growth of a plant, and the outer layer body is formed into a mat shape by inserting many needles into the stacked fiber aggregate from the upper side thereof and pressing the needles.

When many needles are inserted into the stacked fiber aggregate from the upper side thereof, and the aggregate is pressed as described above, horizontal fibers are pressed by the needles to be changed in a longitudinal direction. As compared with a case of simply pressing and forming the fiber aggregate into a mat shape, a fiber layer is realized in which many longitudinal fibers are present. The horizontal and longitudinal fibers are intertwined to be integrated and to be formed into a mat-shaped fiber aggregate of high shape retention difficult to be broken loose. The presence of many longitudinal fibers in the fiber aggregate of the outer layer body facilitates entry of the longitudinal fibers of the outer layer body into the concrete during the forming of the block main body, and the fibers are intertwined well with the block body to be bonded to the outer layer body. Thus, the outer layer body is not easily peeled off from the block main body.

As a result, when the plant grows in the outer layer body, roots of the plant extend in the fiber aggregate, but the rooting of the plant is assured because the outer layer body is not easily peeled off from the block main body. Additionally, in the case of use for the bank protection of the river or the like, easy flowing of the vegetation block is difficult even when it is exposed to a swollen river or a rainstorm, making it possible to secure a satisfactory plant growth environment.

If necessary, natural rubber is sprayed to the mat-shaped fiber aggregate. Accordingly, shape retention is improved. Generally, in the case of a large amount of natural rubber, shape retention is improved while vegetation is reduced. In the case of a small amount of natural rubber, vegetation is secured while shape retention is reduced. According to the invention, however, relatively many longitudinal fibers are present in the fiber aggregate, and the horizontal and longitudinal fibers are intertwined to make breaking-loose difficult. Accordingly, shape retention can be improved even without increasing the amount of natural rubber so much. Thus, shape retention can be improved without reducing vegetation.

According to the invention, the outer layer body is constituted by die-forming the mat-shaped fiber aggregate into a shape similar to that of the outer surface of the block main body. Because of the shape die-formed to be similar to that of the outer surface of the block main body, the shape of the outer layer body can be maintained and shape retention can be improved as compared with a case of simple bonding.

In this case, it is effective to constitute the outer layer body by mixing thermoplastic polymer fibers in the fiber aggregate and then die-forming the mat-shaped fiber aggregate into a shape similar to that of the outer surface of the block main body while heating the same. The mixing-in of the thermoplastic polymer resin fibers facilitates forming, the shape of the outer layer body can be surely maintained after the forming, and shape retention can be improved more.

As aggregates of the concrete, aggregates of uniform grain sizes are used, and a porosity of the concrete is set to be 10 to 25%. The porosity is preferably 15 to 20%.

It is effective to use the aggregates of grain sizes classified within a sieve range of 10 nm to 25 nm as the aggregates. Preferable are the aggregates of grain sizes classified within a sieve range of 13 mm to 20 mm. It is preferred that such aggregates of uniform grain sizes are present in a ratio of 80 vol % or more, preferably 90 vol % or more, more preferably 95 vol % or more of the entire aggregate.

Here, the concrete is a mixture of a binder and the aggregates. Examples of the binder include inorganic and organic binders, which are used singly or mixedly.

Any inorganic binder can be used as long as it is a hydraulic inorganic compound which can harden by reaction with water. For example, inorganic cements, ettringite, plaster and the like are preferable, and the inorganic cements which can easily obtain heat insulation, tenacity and durability are more preferable.

Examples of the inorganic cements include Portland cement, high-early-strength Portland cement, white Portland cement, alumina cement, hydraulic cement such as casting plaster, blast furnace cement, sulfated slag cement, latent hydraulic cement such as lime slag cement, silica cement, mixed cement such as fly-ash cement.

As the organic binder, any can be used as long as it is a reaction hardening type or solidified from a thermoplastic state or the like.

As the aggregate, a general stone material, an artificial lightweight aggregate or an industrial waste may be used. As the artificial lightweight aggregate, crushed plastic is preferred, and plastic foam, or a crushed article such as plastic reduced by melting plastic foam or the like can be used. As the industrial waste, a concrete shell, a concrete sludge, a rubbish melted slug, a cast slug, or cast dusts can be used.

With this configuration, a porosity of the concrete is set relatively large by using the aggregates of uniform grain sizes as the aggregates. Especially, if the aggregates of different grain sizes are mixed, the small and large aggregates are closely boned to each other to make generation of pores difficult. In comparison, in the case of the aggregates of uniform grain sizes, pores are easily generated between the aggregates, and especially continuous pores are easily generated. Thus, during the forming of the block main body, the fibers of the outer layer body easily enter the concrete, and the fibers are intertwined well with the block main body to bond the outer layer body. As a result, together with the effect of the function of bonding to the block main body by the longitudinal fibers of the outer layer body,, the outer layer body is made more difficult to be peeled off easily from the block main body.

When the plant grows in the outer layer body, the roots of the plant extend in the fiber aggregate. However, the rooting of the plant is assured because the outer layer body is not easily peeled off from the block main body.

Furthermore, because of the continuous pores generated in the block main body, the roots of the plant enter the pores to take root in the block main body. Accordingly, the rooting of the plant is assured. Meanwhile, bubbles are generated in foamed concrete to generate pores. However, no roots can enter the pores of the foamed concrete because most bubbles are independently present in the concrete. Thus, the technology of generating continuous pores by using the aggregates of uniform grain sizes is extremely effective for root entry.

As a result, the peeling-off of the outer layer block from the block main body is made more difficult. Thus, in the case of use for the bank protection of the river or the like, easy flowing of the vegetation block is made difficult even when it is exposed to a swollen river or a rainstorm, making it possible to secure a satisfactory plant growth environment.

If necessary, the fiber aggregate contains at least one of plant seeds, a fertilizer, and a water retainer. As the fertilizer, a hardly soluble fertilizer is preferred. As the water retainer, for example, there are paper pulp, water-retaining polymer, and the like. An environment for growing the plant can be secured to facilitate the plant growth. Moreover, a plant suited to the installation environment of the vegetation block can be selected.

In this case, the use of the paper pulp as the water retainer is effective. Absorbability of the pulp can surely improve a water-retaining function.

If necessary, vegetable fibers are used as fibers of the fiber aggregate. Since the vegetable fibers have water absorbance and water retention, and are naturally degradable, an adverse influence on the environment is limited. As the vegetable fibers, wastes such as palm fibers or coconut fibers can also be used.

If necessary, a reinforcing member is embedded in the block main body. The reinforcing member becomes a bone of the block main body to improve strength.

If necessary, the reinforcing member is provided with a connection portion which outwardly projects from the block main body and which is connectable to the adjacent block main body. By interconnecting such connection portions, the vegetation block can be stably installed.

Furthermore, if necessary, the connection portion is formed into a ring shape. A plurality of vegetation blocks can be easily interconnected through rings.

Furthermore, in order to achieve the object, an outer layer body for a vegetation block constituted by forming, into a mat shape, a fiber aggregate which is located on an outer surface of a block main body formed out of concrete during the forming of the block main body and which enables growth of a plant is characterized in that the outer layer body is formed into a mat shape by inserting many needles into the stacked fiber aggregate from the upper side thereof and pressing the needles, and the mat-shaped fiber aggregate is die-formed into a shape similar to that of the outer surface of the block main body.

Thus, as described above, since many needles are inserted into the stacked fiber aggregate from the upper side thereof, and the aggregates is pressed, horizontal fibers are pressed by the needles to be changed in a longitudinal direction. As compared with a case of simply pressing and forming the fiber aggregate into a mat shape, a mat-shaped fiber layer is realized in which many longitudinal fibers are present. The horizontal and longitudinal fibers are intertwined to be integrated and to be formed into a mat-shaped fiber aggregate of high shape retention difficult to be broken loose. The presence of many longitudinal fibers in the fiber aggregate of the outer layer body facilitates entry of the longitudinal fibers of the outer layer body into the concrete during the forming of the block main body, and the fibers are intertwined well with the block body to be bonded to the outer layer body. Thus, the outer layer body is not easily peeled off from the block main body.

As a result, when the plant grows in the outer layer body, roots of the plant extend in the fiber aggregate, but the rooting of the plant is assured because the outer layer body is not easily peeled off from the block main body. Additionally, in the case of use for the bank protection of the river or the like, easy flowing of the vegetation block is difficult even when it is exposed to a swollen river or a rainstorm, making it possible to secure a satisfactory plant growth environment.

The mat-shaped fiber aggregate is die-formed into a shape similar to that of the outer surface of the block main body. Thus, as compared with a simple mat, a shape of the outer layer body can be maintained, making it possible to improve shape retention. Furthermore, for example, when the outer layer body is manufactured at a dedicated forming plant, and then carried to a concrete forming plant located in another place to be formed for the vegetation block, handling is easy, carrying is easy, and concrete forming is easy because the-shape of the outer layer body is maintained.

In this case, it is effective to constitute-the outer layer body by mixing thermoplastic polymer fibers in the fiber aggregate and then die-forming the mat-shaped fiber aggregate into a shape similar to that of the outer surface of the block main body while heating the same. The mixing-in of the thermoplastic polymer resin fibers facilitates forming, the shape of the outer layer body can be surely maintained after the forming, and shape retention can be improved more

If necessary, natural rubber is sprayed to the mat-shaped fiber aggregate. Accordingly, shape retention is improved more. Generally, in the case of a large amount of natural rubber, shape retention is improved while vegetation is reduced. In the case of a small amount of natural rubber, vegetation is secured while shape retention is reduced. According to the invention, however, relatively many longitudinal fibers are present in the fiber aggregate, and the horizontal and longitudinal fibers are intertwined to make breaking-loose difficult. Accordingly, shape retention can be improved even without increasing the amount of natural rubber so much. Thus, shape retention can be improved without reducing vegetation.

If necessary, the fiber aggregate contains at least one of plant seeds, a fertilizer, and a water retainer. As the fertilizer, a hardly soluble fertilizer is preferred. As the water retainer, for example, there are paper pulp, water-retaining polymer, and the like. An environment for growing the plant can be secured to facilitate the plant growth. Moreover, the plant suited to the installation environment of the vegetation block can be selected.

In this case, it is effective to use the paper pulp as the water retainer. Absorbability of the pulp can surely improve a water-retaining function.

The paper pulse is preferably contained in the die-formed outer layer body by dipping the outer layer body in a paper pulp liquid. In this case, the fertilizer and the plant seeds can be mixed in the paper pulp. They can be mixed therein prior to the formation of the outer layer body, but there is a danger that a plant germination rate is lowered by heating. In the case of spraying natural rubber, it is considered that the natural rubber is partially adsorbed by the fertilizer and the water retainer. In addition, there is also a fear that functions of the fertilizer and the water retainer are reduced. Therefore, a method is preferable wherein the outer layer body is first formed, and the thus formed outer layer body is then dipped in the paper pulse solution including the fertilizer and the plant seeds.

Furthermore, if necessary, vegetable fibers are used as fibers of the fiber aggregate. Since the vegetable fibers have water absorbance and water retention, and are naturally degradable, an adverse influence on the environment is limited. As the vegetable fibers, wastes such as palm fibers or coconut fibers can also be used.

### BRIEF DESCRIPTION OF THE DRAWINGS

FIGS. 1(a) and 1(b) show an example of a vegetation block according to an embodiment of the present invention; and FIG. 1(a) is a perspective view and FIG. 1(b) is a sectional view.

FIG. 2 is concerned with a method of manufacturing the vegetation block according to the embodiment of the present invention, and shows an outer layer body forming process of manufacturing an outer layer body for the vegetation block according to the embodiment of the present invention, and in particular, it is a perspective view showing a step (1-1) wherein of many needles are inserted into a stacked fiber aggregate from the upper side thereof and then pressed to form the fiber aggregate into a mat shape.

FIG. 3 is a side view showing the step (1-1) in the outer layer body forming process wherein many needles are inserted into the stacked fiber aggregate from the upper side thereof and then pressed to form the fiber aggregate into the mat shape.

FIG. 4 shows a step (1-2) in the outer layer body forming process wherein natural rubber is sprayed to the cut mat-shaped fiber aggregate.

FIG. 5 shows a step (1-3) in the outer layer body forming process wherein the cut mat-shaped fiber aggregate is die-formed.

FIG. 6 shows a step (1-4) in the outer layer body forming process wherein the die-formed outer layer body is dipped in a paper pulp liquid containing plant seeds and a fertilizer.

FIG. 7 is concerned with a method of forming the vegetation block according to the embodiment of the present invention, and describes a process of forming the vegetation block by using the vegetation block outer layer body according to the embodiment of the present invention, and in particular, it shows a step (2-1) of attaching the outer layer body to a forming die and a step (2-2) of receiving a reinforcing member in the forming die.

FIGS. 8(a) and 8(b) show a step (2-3) wherein the forming die is mounted on a vibration press type forming machine and concrete to be formed is poured; and FIG. 8(a) shows a state of pouring the concrete and FIG. 8(b) shows its state during the forming.

FIGS. 9(a) and 9(b) show a last state of the forming process; and FIG. 9(a) shows a step (2-4) wherein the forming die is taken out of the forming machine and the concrete cures, and FIG. 9(b) shows a release step (2-5) of the concrete from the forming die after the curing.

FIG. 10 shows a state of a bonded block main body and the outer layer body in the vegetation block according to the embodiment of the present invention.

FIG. 11 is a perspective view showing an example of a conventional vegetation block.

### BEST MODE FOR CARRYING OUT THE INVENTION

Hereinafter, a vegetation block according to an embodiment of the present invention and an outer layer body for the vegetation block will be described with reference to the accompanying drawings.

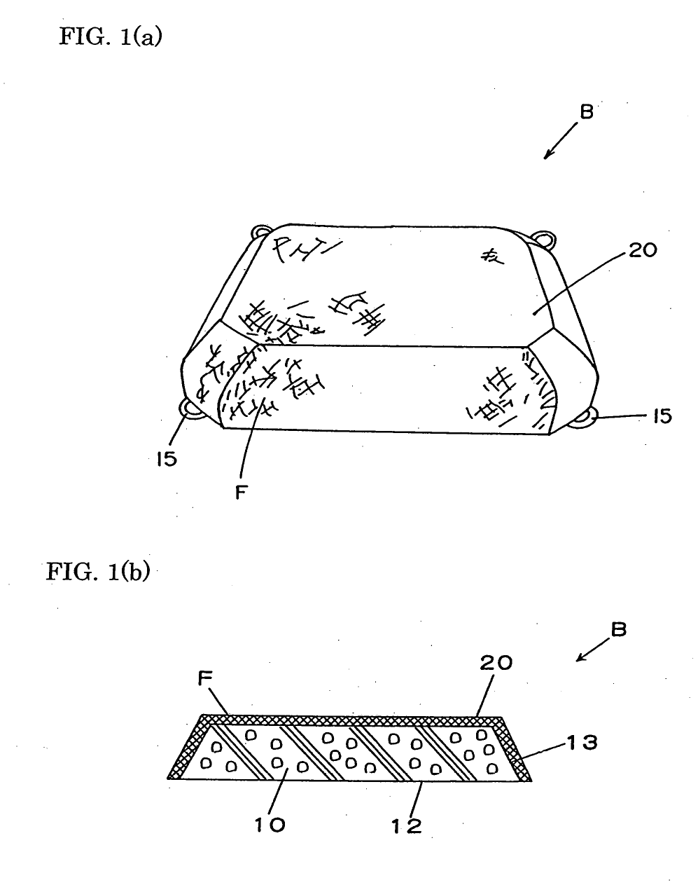

As shown in FIGS. 1(a) and 1(b), a vegetation block B is configured by comprising a block main body 10 having a bottom surface 12 which is installed on an installation surface made of concrete, and an outer layer body 20 constituted by forming, into a mat shape, a fiber aggregate F which is located on an outer surface 13 excluding the bottom surface 12 of the block main body 10 during the forming of the block main body 10 and which enables growth of a plant.

The block main body 10 may take any shape as long as an area exposed to the outside is large. For example, it may be formed into a rectangular parallelepiped shape, a roughly semispherical shape, a conical shape, a truncated conical shape or the like. According to the embodiment, the block main body 10 is formed into a roughly square truncated conical shape in which corners are chamfered.

As shown in FIG. 7, metal reinforcing members 14 are embedded in the block main body 10. The reinforcing members 14 comprise connection portions 15 in which two steel wires cross at a centroidal place of the block main body 10 to contact each other and to outwardly project from the block main body 10. A plurality of connection portions 15 constituted by forming steel wires into ring shapes are disposed at a relation of equal angles. According to the embodiment, the block main body 10 is disposed at each corner.

The concrete of the block main body 10 is prepared by mixing a binder with an aggregate K (FIG. 10). As the binder, there are inorganic and organic binders which are independently used or mixed. According to the embodiment, inorganic cement as a hydraulic inorganic binder hardened by reacting with water is used.

As the binder, a general stone, an artificial lightweight aggregate, or an industrial waste is used. The aggregate may be spherical or heteromorphic, but an aggregate of a uniform grain size is preferably used. Specifically, the aggregates of grain sizes classified within a sieve range of 10 mm to 25 mm, more preferably the aggregates of grain sizes classified within a sieve range of 13 mm to 20 mm, are used. Such an aggregate of a uniform grain size is preferably contained in an amount of 80 vol % or more, preferably 90 vol % or more, and more preferably by 95 vol % or more of the entire aggregates.

For example, a mixing ratio between the binder and the aggregate is that the inorganic binder is 270 Kg/m³ and the aggregate is 1700 Kg/m³.

Accordingly, a concrete porosity is 10 to 25%, preferably 15 to 20%.

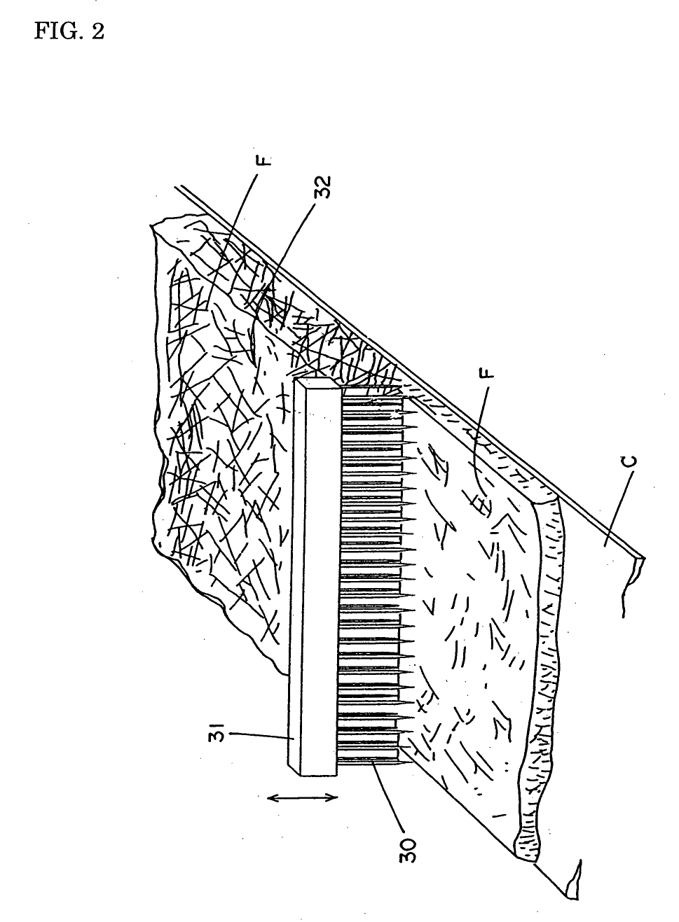

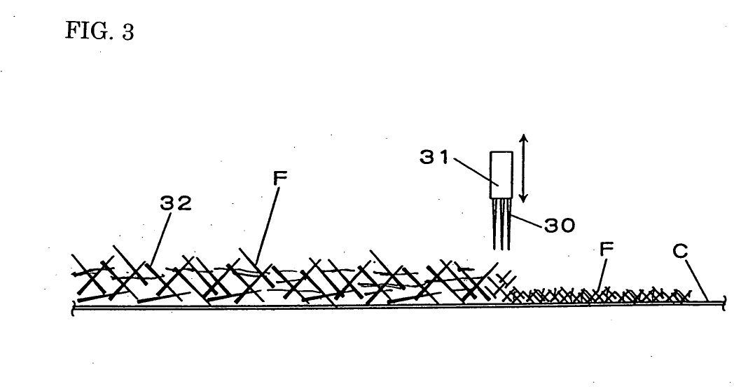

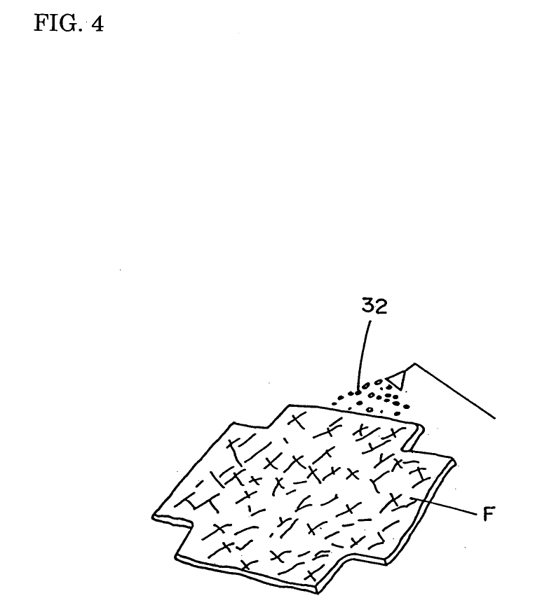

The outer layer body 20 of the vegetation block according to the embodiment of the invention comprises the fiber aggregate F constituted of palm fibers which are vegetable fibers. As shown in FIGS. 2 and 3, the outer layer body 20 is formed into a mat shape by inserting many needles 30 planted in a support member 31 into the stacked fiber aggregate F from the upper side thereof and then pressing the needles. As shown in FIG. 4, natural rubber 32 is sprayed to the mat-shaped fiber aggregate F. Additionally, the outer layer body 20 is constituted by mixing thermoplastic polymer fibers 33 in the fiber aggregate F, and subjecting the mat-shaped fiber aggregate F to die-forming while heating it into a shape of the outer surface 13 excluding the bottom surface 12 of the block main body 10.

For the thermoplastic polymer fibers, for example, polyethylene (PE), polystyrene (PS), or an acrylonitrile/butadiene/styrene resin (ABS) is used. Biodegradable plastic is preferable because it can reduce a load on an environment. A fiber length is 20 to 150 mm, preferably 30 to 100 mm, and more preferably 50 to 100 mm. It is because if combined with lengths of vegetable fibers, uniform dispersion of the fibers is facilitated during kneading.

For a mixing amount of the thermoplastic polymer fibers, its volume ratio in the entire fiber aggregate volume is 1% to 15%, preferably 3% to 10%, and more preferably 3% to 5%.

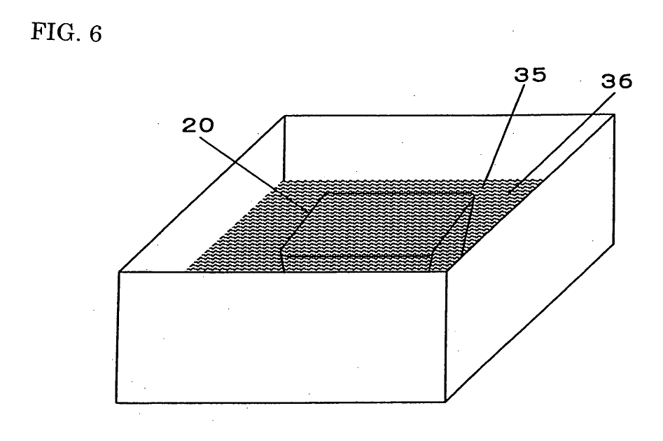

Furthermore, the outer layer body 20 contains plant seeds, a fertilizer, and a water retainer. For the plant seeds, desired seeds can be used in accordance with an installation environment. The fertilizer can be used in accordance with a plant to be grown. For the water retainer, there are various types such as water-retaining polymer which can retain water. According to the embodiment, paper pulp 35 (see FIG. 6) is used.

Next, a method of manufacturing the vegetation block B of the embodiment will be described with reference to FIGS. 2 to 10. This manufacturing method comprises (1) an outer layer body forming step of forming the outer layer body 20 of the vegetation block of the embodiment of the invention, and (2) a forming step of forming the block main body 10 together with the outer layer body 20. The method will be described in detail hereinafter.

(1) Outer Layer Body Forming Step
(1-1)

As shown in FIGS. 2 and 3, palm fibers are stacked roughly with a predetermined thickness on a conveyor C to be conveyed. In this conveying process, many needles 30 are inserted into the stacked fiber aggregate from the upper side thereof, and the fiber aggregate F is pressed to be formed into a mat shape. Thermoplastic polymer fibers 32 have been mixed in the palm fibers.

Many needles 30 are planted downward in a matrix in the support member 31 extended in a width direction of the conveyor C. This support member 31 is reciprocated up and down by a driving mechanism (not shown). Many needles 30 are inserted into the fiber aggregate F from the upper side thereof which is stacked on the conveyor C to be conveyed thereon, and the fiber aggregate is pressed.

Accordingly, the mat-shaped fiber aggregate F is formed. In this case, many needles 30 are inserted into the stacked fiber aggregate F from the upper side thereof, and the fiber aggregate is then pressed. Thus, horizontal fibers are pressed by the needles 30 to be changed in a longitudinal direction. As compared with the case of simply pressing and forming a fiber aggregate into a mat shape, many longitudinal fibers are present to form a mat-shaped fiber layer, and the horizontal and longitudinal fibers are intertwined to be integrated, and formed into a mat-shaped fiber aggregate F difficult to be broken loose and having high shape retention. Thus, there are many longitudinal fibers in the fiber aggregate F of the outer layer body 20.

(1-2)

As shown in FIG. 4, the mat-shaped fiber aggregate F is cut into a rectangular shape by a cutting machine, and each corner is cut into a rectangular shape. Then, liquid natural rubber 32 is sprayed to front and back surfaces of the fiber aggregate, and these surfaces are dried.

(1-3)

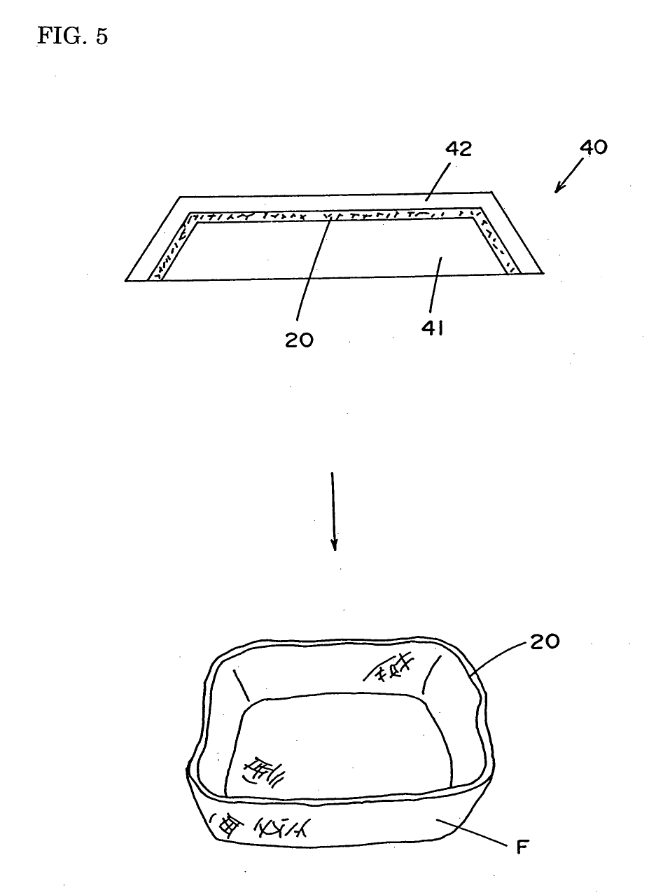

Next, as shown in FIG. 5, the cut mat-shaped fiber aggregate F is subjected to die-forming. A forming die 40 comprises a lower die 41 and an upper die 42 having a die surface similar to a shape of the outer surface 13 excluding the bottom surface 12 of the block main body 10. To facilitate the forming, steam is fed into the upper die 42 and the lower die 41 to heat them. Then, the mat-shaped fiber aggregate F is placed into the lower die 41 and pressed by the upper die 42. In consequence, the mat-shaped fiber aggregate F is die-formed into the shape similar to that of the outer surface 13 excluding the bottom surface 12 of the block main body 10, while heated. In this case, owing to the mixing of the thermoplastic polymer resin fibers 32, the forming can be facilitated, a shape of the outer layer body 20 can be surely maintained after the forming, and a shape retaining performance can be improved.

(1-4)

Next, as shown in FIG. 6, the die-formed outer layer body 20 is dipped in a paper pulp liquid 36 containing plant seeds and a fertilizer to include the plant seeds, the fertilizer and the paper pulp 35 as the water retainer in the fiber aggregate F. Then, the aggregate is dried, whereby the outer layer body 20 of the vegetation block according to the embodiment of the present invention is manufactured.

(2) Forming Step

(2-1)

As shown in FIG. 7, a forming die 50 having an opened top part is prepared in accordance with the shape of the block main body 10. The forming die 50 having the opened top part is formed by welding a metal plate material. The die-formed outer layer body 20 is received in this forming die 50.

(2-2)

In this state, as shown in FIG. 7, a reinforcing member 14 is fixed in the forming die 50. The reinforcing member 14 is disposed so that a connection portion 15 may outwardly project from the forming die 50.

(2-3)

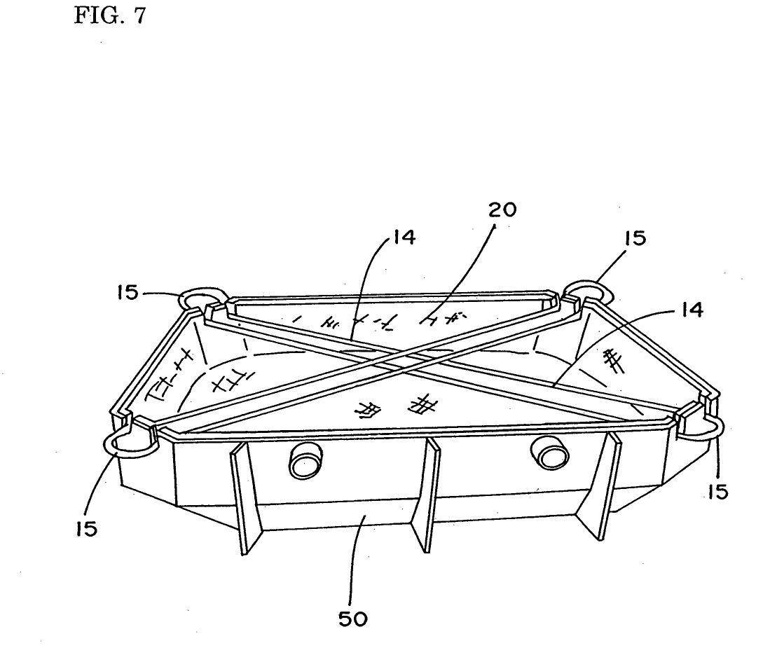

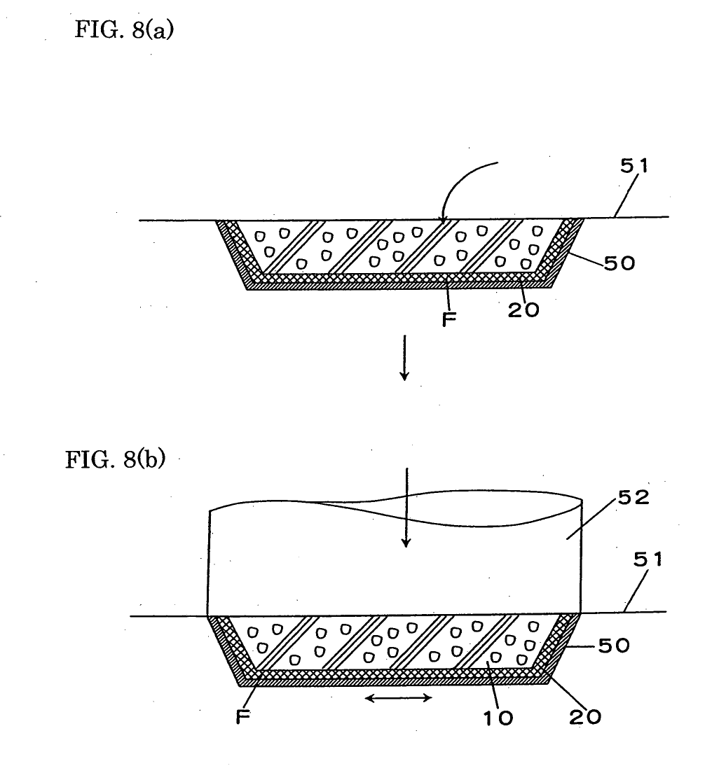

Next, as shown in FIG. 8(a), for example, the forming die 50 is mounted on a vibration press type forming machine 51, and concrete is then poured thereinto. In this state, as shown in FIG. 8(b), an exposed upper surface of the poured concrete is pressed by a pressing die 52 while vibration is applied to the forming die 50. By such operations of applying the vibration to the forming die 50 and pressing the concrete surface with the pressing die 52, a concrete density of the block main body 10 is increased to improve the strength of the concrete.

(2-4)

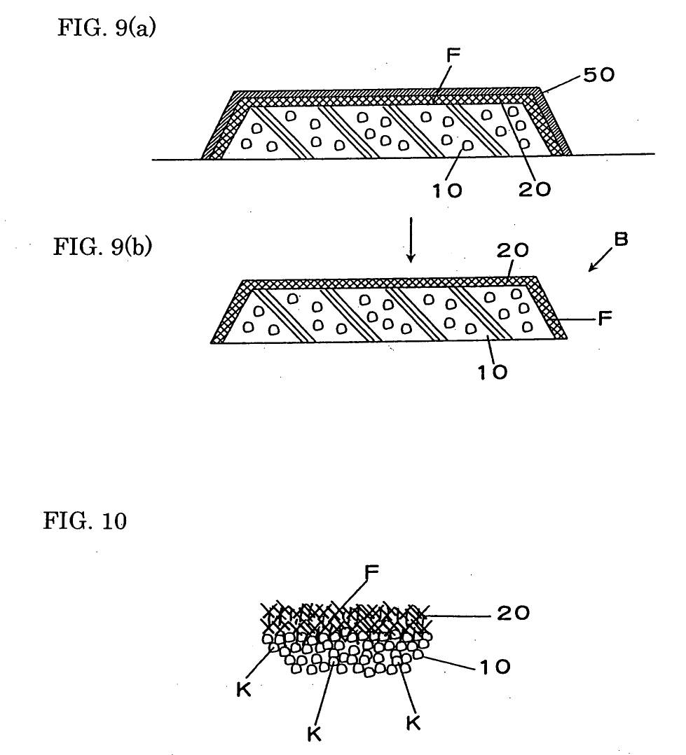

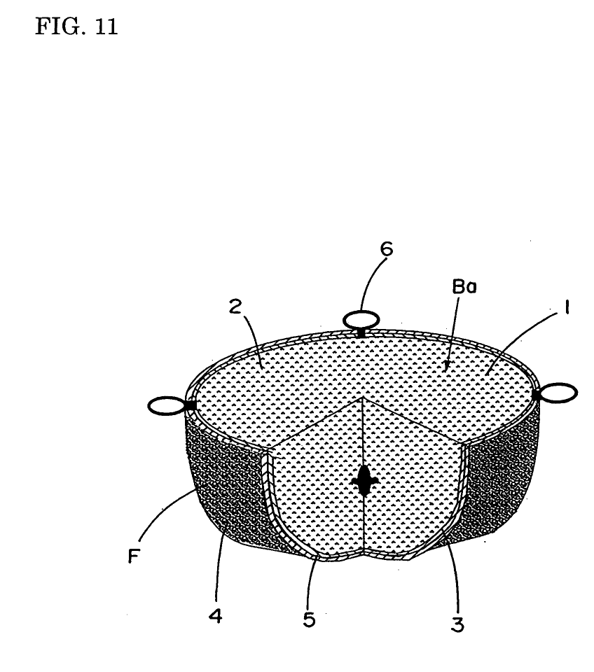

Then, as shown in FIG. 9(a), the forming die 50 is reversed and taken out from the vibration press type forming machine 51, and the concrete is allowed to cure for a required time. In consequence, the concrete hardens while bonding to the outer layer body 20. In this case, as shown in FIG. 10, an aggregate of a uniform grain size is used as the aggregate K, so that a porosity of the concrete increases. In contrast, if the aggregates of different grain sizes are used, the large and small aggregates closely bond to each other, thereby making the generation of pores difficult. However, when the aggregate of the uniform grain size is used, the pores are easily generated among the aggregates, so that the fibers of the outer layer body 20 can easily enter into the pores of the concrete. In consequence, the fibers are well intertwined with the block main body 10 and therefore the outer layer body 20 bonds to the block main body 10, whereby the outer layer body 20 is not easily peeled off form the block main body 10.

Further, in this case, since many longitudinal fibers are present in the fiber aggregate F of the outer layer body 20, the longitudinal fibers of the outer layer body 20 can easily enter the concrete. In consequence, the fibers are well intertwined with the block main body 10 and therefore the outer layer body 20 bonds to the block main body 10. Also owing to this constitution, the outer layer body 20 is not easily peeled off form the block main body 10.

(2-5)

Lastly, as shown in FIG. 9(b), the cured concrete is released from the forming die, whereby the vegetation block B is manufactured.

In this vegetation block B, the fibers of the outer layer body 20 well enter the pores of the concrete, so that they are well intertwined with the block main body 10 and therefore the outer layer body 20 bonds to the block main body 10, whereby the outer layer body 20 is not easily peeled off form the block main body 10. In addition, the longitudinal fibers of the outer layer body 20 enter the concrete, and they are well intertwined with the block main body 10, so that the outer layer body 20 bonds to the block main body 10. Also owing to this constitution, the outer layer body 20 is not easily peeled off form the block main body 10.

Furthermore, in the fiber aggregate F, the relatively plentiful longitudinal fibers are present, so that the horizontal and longitudinal fibers are intertwined with each other and hence these fibers do not easily loosen, whereby the shape of the fiber aggregate F can be maintained. In addition, the natural rubber is sprayed to the outer layer body 20, and hence the shape maintenance of the fiber aggregate F can further be improved. Furthermore, the outer layer body 20 is die-formed into the shape similar to that of the outer surface 13 of the block main body 10, and it is mixed with the thermoplastic highly polymer resin fibers. In consequence, the shape of the outer layer body 20 can be surely maintained, whereby a shape maintenance effect can be improved.

Furthermore, the reinforcing member 14 is embedded in the block main body 10, and hence the reinforcing member 14 becomes a bone of the block main body 10 to improve the strength of the block main body 10.

Next, when the vegetation block B of the embodiment is used for, e.g., a bank protection of a river, the bottom surfaces 12 of the vegetation blocks B are brought into contact with an installation surface, and the connection portions 15 are interconnected to each other to install the vegetation blocks B. In this case, since the connection portions 15 are mutually interconnected, the vegetation blocks B are stably installed. Since the connection portions 15 are formed into a ring shape, a plurality of vegetation blocks B can easily be interconnected through the rings.

In the state of the installed vegetation blocks B, the outer layer bodies 20 contain the plant seeds, the fertilizer and the water retainer, and hence the seeds germinate and grow with the aid of the fertilizer. In this case, the presence of the water retainer prevents the seeds from drying, and so the growth of the plant is easy. Especially, since the water retainer is paper pulp, its absorbability surely increases a water retaining function. Additionally, since the outer layer bodies 20 are constituted of the palm fibers, the vegetation blocks B are excellent in water absorbing properties and water retaining properties. In addition, since the vegetation blocks B is naturally degradable, an adverse influence on an environment is limited.

As the plant grows in the outer layer bodies 20, the roots of the plant extend in the fiber aggregates F. However, the outer layer bodies 20 do not easily peel off from the block main bodies 10, and hence the rooting of the plant surely proceeds. Since the continuous pores are especially formed in the block main bodies 10, the roots of the plant enter the pores and extend even in the block main bodies 10. Also owing to this constitution, the rooting of the plant securely proceeds.

Furthermore, the outer layer bodies 20 do not easily peel off from the block main bodies 10, and hence even when the fiber aggregates are used for the bank protection of the river and the outer layer bodies are exposed to increased water of the river or a rainstorm, the outer layer bodies do not easily run off. In consequence, a satisfactory plant growth environment can sufficiently be secured.

It is to be noted that the shape of the block main bodies 10 of the vegetation blocks B according to the above embodiment is not limited to the above shape, and the block main body 10 can be formed into an optional shape such as a polygonal cone, a circular cone or a truncated cone.

### INDUSTRIAL APPLICABILITY

As described above, the vegetation block and the outer layer body for the vegetation block of the present invention can effectively be used on a slope along a road, a bank protection or the like.

1. A vegetation block comprising a block main body formed out of concrete; and an outer layer body constituted by forming, into a mat shape, a fiber aggregate which is located on an outer surface of the block main body during the forming of the block main body and which enables growth of a plant,
characterized in that the outer layer body is formed into a mat shape by inserting many needles into the stacked fiber aggregate from the upper side thereof and pressing the needles.

2. The vegetation block according to claim 1, characterized in that natural rubber is sprayed to the mat-shaped fiber aggregate.

3. The vegetation block according to claim 1, characterized in that the outer layer body is constituted by die-forming the mat-shaped fiber aggregate into a shape similar to that of the outer surface of the block main body.

4. The vegetation block according to claim 1, characterized in that the outer layer body is constituted by mixing thermoplastic polymer fibers in the fiber aggregate and then die-forming the mat-shaped fiber aggregate into a shape similar to that of the outer surface of the block main body while heating the same.

5. The vegetation block according to claim 1, characterized in that the aggregates of uniform grain sizes are used as the aggregates of the concrete, and a porosity of the concrete is set to be 10 to 25%.

6. The vegetation block according to claim 5, characterized in that the aggregates of grain sizes classified within a sieve range of 10 nm to 25 nm are used as the aggregates.

7. The vegetation block according to claim 1, characterized in that the fiber aggregate contains at least one of plant seeds, a fertilizer, and a water retainer.

8. The vegetation block according to claim 7, characterized in that paper pulp is used as the water retainer.

9. The vegetation block according to claim 1, characterized in that vegetable fibers are used as fibers of the fiber aggregate.

10. The vegetation block according to claim 1, characterized in that a reinforcing member is embedded in the block main body.

11. The vegetation block according to claim 10, characterized in that the reinforcing member is provided with a connection portion which outwardly projects from the block main body and which is connectable to the adjacent block main body.

12. The vegetation block according to claim 11, characterized in that the connection portion is formed into a ring shape.

13. An outer layer body for a vegetation block constituted by forming, into a mat shape, a fiber aggregate which is located on an outer surface of a block main body formed out of concrete during the forming of the block main body and which enables growth of a plant,
the outer layer body being formed into a mat shape by inserting many needles into the stacked fiber aggregate from the upper side thereof and pressing the needles, the mat-shaped fiber aggregate being die-formed into a shape similar to that of the outer surface of the block main body.

14. The outer layer body for the vegetation block according to claim 13, wherein the outer layer body is constituted by mixing thermoplastic polymer fibers in the fiber aggregate and then die-forming the mat-shaped fiber aggregate into a shape similar to that of the outer surface of the block main body while heating the same.

15. The outer layer body for the vegetation block according to claim 13, characterized in that natural rubber is sprayed to the mat-shaped fiber aggregate.

16. The outer layer body for the vegetation block according to claim 13, characterized in that the fiber aggregate contains at least one of plant seeds, a fertilizer, and a water retainer.

17. The outer layer body for the vegetation block according to claim 16, characterized in that paper pulp is used as the water retainer.

18. The outer layer body for the vegetation block according to claim 13, characterized in that vegetable fibers are used as fibers of the fiber aggregate.
